(19) United States
(12) Patent Application Publication
(10) Pub. No.: US 2017/0159096 A1
(43) Pub. Date: Jun. 8, 2017

(54) METHOD FOR FUSION EXPRESSION OF ION CHANNEL PROTEIN AND TRANSPORT PROTEIN AND PROTEIN FRAGMENT USED THEREFOR

(71) Applicant: VIVA Biotech (Shanghai) Ltd., Shanghai (CN)

(72) Inventors: Dongming Qian, Shanghai (CN); Jianhua Cai, Shanghai (CN); Liang Hu, Shanghai (CN); Ping Wang, Shanghai (CN); Lirong Wang, Shanghai (CN); Xiaobo Yu, Shanghai (CN); Danjing Wang, Shanghai (CN); Xiuhong Zeng, Shanghai (CN); Chen Mao, Shanghai (CN); Delin Ren, Shanghai (CN)

(73) Assignee: VIVA Biotech (Shanghai) Ltd.

(21) Appl. No.: 15/362,826

(22) Filed: Nov. 29, 2016

Related U.S. Application Data
(63) Continuation of application No. PCT/CN2014/085285, filed on Aug. 27, 2014.

(30) Foreign Application Priority Data
Jun. 5, 2014 (CN) 201410247582.3

(51) Int. Cl. C12P 21/02; C07K 14/47

(52) U.S. Cl. CPC C12P 21/02; C07K 14/47; C07K 2319/00

(57) ABSTRACT

The present invention provides a protein fragment for fusion expression of ion channel protein and transport protein. The protein fragment is a Bril protein fragment or a T4L protein fragment. Also provided is a method for preparing a fusion protein by inserting a Brit protein fragment or T4L protein fragment into the N-terminal, C-terminal or intramembrane loop region of the ion channel protein and transport protein so as to improve its, in-vitro stability and crystallizability.

### CROSS-REFERENCE TO RELATED APPLICATIONS

This application is a continuation of International Patent Application No. PCT/CN2014/085285 with a thing date of Aug. 27, 2014, designating the United States, now pending, and further claims priority to Chinese Patent Application No. 201410247582,3 with a filing date of Jun. 5, 2014. The content of the aforementioned application, including any intervening amendments thereto, are incorporated herein by reference.

### TECHNICAL FIELD

The present invention relates to the genetic, engineering field, in particular, the present invention relates to a method for fusion expression of ion channel protein and transport protein as well as protein fragment used therefor.

### BACKGROUND OF THE PRESENT INVENTION

Ion channel protein is a pore-forming protein that allows certain specific types of ions to pass through the channel by their electrochemical gradient so as to help cells to constitute and control slight voltage difference between plasma membranes. These ion channels are present in cell membrane of all cells. Ion channels can be divided into chloride ion channel family, potassium ion channel family, sodium ion channel family, calcium ion channel family, proton channel family and universal ion channel family in accordance with types of ions.

Transport protein is capable of selectively allowing non-free diffusion small molecular substances to penetrate plasma membrane. Transport protein is capable of allowing a vast majority of molecules and inorganic ions that are not soluble in lipids and are important in metabolism to effectively enter and exit the viable cells and has a fine control mechanism, so as to make them have a suitable concentration gradient inside and outside of a cell, thereby forming, a certain membrane potential difference. Transport proteins can be divided into the three classes in accordance with mode of action thereof: carrier proteins, channel proteins, and ion pumps.

Since voltage-activated channels are the basis of nerve impulses and conduction-activated channels are responsible for signal transduction among synapses, the ion channel protein and transport protein play an important role in the nervous, system. In fact, aggressive or defensive neurotoxins of a most majority of organisms all achieve the purpose of shutting down or paralyzing the system by adjusting the conductivity and kinetic characteristics of channels. In addition, the ion channel protein and transport protein are also a critical structure for a large number of biological processes involved in rapid changes in cells, for example, contraction of cardiac muscle, skeletal muscle and smooth muscle, transmission of nutrients and ions to the epithelium, activation of T cells, and release of insulin from pancreatic beta-cells. In the study of new drugs, the ion channel protein and transport protein are a very common goal to be researched.

The study on structure and function of ion channel protein and transport protein has been a very important research hotspot and difficulty due to their important physiological functions in vivo. However, natural ion channel protein and transport protein are not stable in vitro, and it is difficult to obtain stable ion channel protein and transport protein with a high purity.

In recent years, it has been reported by several research groups that ion channel proteins of different species and corresponding crystal structures thereof are obtained from prokaryotic expression systems by means of purification. The current research is focused on ion channel protein and transport protein from prokaryotes, and expression purification and crystallization of human ion channel protein and transport protein have rarely been reported at home and abroad. The main reason is that ion channel protein and transport protein have quite bad solubility and low stability, and a large number of stable proteins could hardly be obtained by in vitro recombinant expression for research. The present invention provides a method for fusion expression of ion channel protein and transport protein, which can be applied to solve the problems associated with the stability and high-expression in vitro of human ion channel protein and transport protein.

### SUMMARY OF PRESENT INVENTION

The invention aims to overcome the above defects, to solve the problems involved in recombinant expression of ion channel protein and transport protein, and to provide a technical platform for researching in vitro expression purification and crystallization of ion channel protein and transport protein, and to lay the foundation for studying the structure of ion channel protein and transport protein and for researching and developing drugs with targeting ion channel protein and transport protein.

The present application provides a novel method for fusion expression of ion channel protein and transport protein by using a BRIL or T4L protein fragment.

In particular, the present invention provides a protein fragment for fusion expression of ion channel protein and transport protein, characterized in that the protein fragment is a Bril protein fragment (amino acid sequence 23-128, protein number: P0ABE7) or a protein having a sequence homology of greater than 90% with this fragment, or a T4L protein fragment (amino acid sequence 2-161, protein number: P00720) or a protein having a sequence homology, of greater than 90% with this fragment.

Wherein, the said Bril protein fragment or the protein having a sequence homology of greater than 90% with this fragment has an amino acid sequence as set forth in SEQ ID NO.1.1; and the said T4L protein fragment or the protein having sequence homology of greater than 90% with this fragment has an amino acid sequence as set forth in SEQ ID NO.1.2.

In addition, the present invention also provides a coding gene sequence, as set forth in SEQ ID NO.2.1, of the Bril protein fragment or the protein having a sequence homology of greater than 90% with this fragment, and a coding gene sequence, as set forth in SEQ ID NO.2.2, of the T4L, protein fragment or the protein having a sequence homology of greater than 90% with this fragment.

Moreover, the present invention also provides a method for fusion expression of ion channel protein and a transport protein, characterized by inserting the above amino add sequence of the protein fragment into the N-terminal, C-terminal or intramembrane loop region of the ion channel protein or transport protein to construct a fusion protein.

Preferably, ASIC (acid-sensing ion channel) GLUTI (glucose transporter) are used.

Specifically, the present invention provides the application of ion channel fusion expression protein comprising a sequence as set forth in SEQ ID NO.1.1 and a sequence as set forth in SEQ ID NO.1.2 to ASIC (acid-sensing ion channel) and GLUTI (glucose transporter), that is, the above-described protein fragment, such as BRIL and T4L, is inserted into different regions (N-terminal, C-terminal or intramembrane loop region) of the ASIC (acid-sensing ion channel) and the GLUT1 (glucose transporter) to construct a fusion protein.

Furthermore, namely, the above-described Bril protein fragment and ASIC are subjected to fusion expression to obtain a fusion protein having an amino acid sequence as set forth in SEQ ID NO.3.1; or the above-described T4L protein fragment and ASIC are subjected to fusion expression to obtain a fusion protein having an amino acid sequence as set forth in SEQ ID NO.3.2.

Also, the ASIC fusion expression plasmid constructed by using the above-described Bril protein fragment has a protein sequence as set forth in SEQ ID NO.4.1, or the ASIC fusion expression plasmid constructed by using above T4L protein fragment has a protein sequence as set forth in SEQ ID NO.4.2.

In addition, the above-described Bril protein fragment and GLUT1 are subjected to fusion expression to obtain a fusion protein having an amino acid sequence as set forth in SEQ ID NO.3.3; or the above-described T4L protein fragment and GLUT1 are subjected to fusion expression to obtain a fusion protein having an amino acid sequence as set forth in SEQ ID NO.3.4.

Also, the GLUT1 fusion expression plasmid constructed by using the above-described Bril protein fragment has a protein sequence as set forth in SEQ ID NO.4.3; or the GLUT1 fusion expression plasmid constructed by using the above-described T4L protein fragment has a protein sequence as set forth in SEQ ID NO.4.4.

Furthermore, it has been found in the present invention that the above-described human ASIC (acid-sensing ion channel) fusion expression plasmid can be expressed in insect cells (e.g., SF9, SF21, HiFive, etc.), the human GLUT1 (glucose transporter) fusion expression plasmid can be expressed in mammalian cells (e.g., 293, CHO, etc.).

The gene sequences as set forth in SEQ ID NO.4.1, SEQ ID NO.4.2, SEQ ID NO.4.3, SEQ ID NO.4.4 are constructed into an expression vector, such as pFastBac1, PcDNA3.1, PET21b and the like, for protein expression.

Usage and Effect of the Present Invention:

In order to improve the soluble expression of ion channel protein and transport protein and the stability of the proteins, in the present invention, a target ion channel protein or transport protein is uniquely modified, that is, a fusion protein expression plasmid is constructed by attempting to insert a protein fragment such as BRIL or T4L etc. into the N-terminal, C-terminal or intramembrane loop, area of the ion channel protein and transport protein so as to improve the in-vitro stability and crystallizability thereof, whereby an ion channel protein and a transport protein having good stability and high expression level and being crystallizable is successfully obtained.

This kind of protein is a very important drug target protein, and the study on structure of this kind of protein will help the research and development of drugs targeting ion channel protein and transport protein in the future. It is worth noting that the method according to the present invention can be widely applied to the research of various ion channel protein and transport protein.

It has been found after research that the fusion protein constructed in the present invention can be expressed in insect cells and can also be expressed in yeast cells and mammalian cells and so on, and has wide application scope.

### DESCRIPTION OF THE DRAWINGS

FIG. 1 the electropherograms of the two purified ASIC1 fusion proteins, wherein the left spectrum is for the T4L-ASIC fusion protein, and the right spectrum is for the BRIL-ASIC1 fusion protein.

FIG. 2 the UPLC chromatograms of the two ASIC1 fusion proteins, wherein the left spectrum is for the T4L-ASIC fusion protein, and the right spectrum is for the BRIL-ASIC1 fusion protein.

FIG. 3 the electropherograms of the two purified GLUT1 fusion proteins, wherein the left spectrum is for the T4L-GLUT1 fusion protein, and the right spectrum is for the BRIL-GLUT1 fusion protein.

FIG. 4 the UPLC chromatograms of the two purified ASIC1 fusion proteins, wherein the left spectrum is for the T4L-GLUT1 fusion protein, and the right spectrum is for the BRIL-GLUT1 fusion protein.

### DETAILED DESCRIPTION OF PREFERRED EMBODIMENTS

A person skilled in the art will be able to construct a plasmid for fusion expression of ion channel protein or transport protein comprising a BRIL or T4L protein fragment by using methods well-known in the art, and these methods include in vitro recombination of DNA techniques, DNA synthesis techniques, in vivo recombination techniques. The constructed gene sequence for fusion expression of ion channel protein and transport protein can be effectively connected to an appropriate promoter of different expression vectors to guide the synthesis of mRNA.

The constructed plasmids can be transfected or transformed into cells by using conventional techniques well known to a person skilled in the art, for example, the PFastBac vector containing the fusion expression gene can be transformed into SF9 cells using the Bac-to-Bac technique for expression. Culture of the transformed cells and collection and passage of viruses, etc., are conventional techniques well known to a person skilled in the art.

### EXAMPLE 1

### Synthesis of Gene of ASIC1 (Acid-Sensing Ion Channel 1) Fusion Protein

Chemical synthesis

BRIL protein fragment (amino acid sequence as set forth in SEQ ID NO.1.1), nucleotide template (SEQ ID NO.2.1)

(5′ GCTGATCTGGAAGACAATTGGGAAACTCTGAACGACAATCTCAAGG
TGATCGAGAAGGCTGACAATGCTGCACAAGTCAAAGACGCTCTGACCAAG
ATGAGGGCAGCAGCCCTGGACGCTCAGAAGGCCACTCCACCTAAGCTCGA
GGACAAGAGCCCAGATAGCCCTGAAATGAAAGACTTTCGGCATGGATTCG
ACATTCTGGTGGGACAGATTGATGATGCACTCAAGCTGGCCAATGAAGGG
AAAGTCAAGGAAGCACAAGCAGCCGCTGAGCAGCTGAAGACCACCCGGAA
TGCATACATTCAGAAGTACCTG 3′),
the forward primer was:
(5′ CCCACCATCGGGCGCGGATCCATGCATCACCATCATCACCACCATC
ACGCTGATCTGGAAGACAATTGG 3′),
the reverse primer was:
(5′ CCCCTGAAAGTACAGGTTTTCCAGGTACTTCTGAATGTATGC
3′),

the gene of BRIL protein fragment was obtained by means of PCR reaction.

The ASIC1 nucleotide template was chemically synthesized with reference to human gene codons,

(5′ ATGGAACTGAAGGCCGAGGAGGAGGAGGTGGGTGGCGTCCAGCCGG
TGAGCATCCAGGCCTTCGCCAGCAGCTCCACACTGCACGGCCTGGCCCAC
ATCTTCTCCTACGAGCGCCTGTCTCTGAAGCGCGCACTGTGGGCCCTGTG
CTTCCTGGGCTCCCTGGCTGTGCTGCTGTGTGTGTGCACGGAGCGCGTGC
AGTACTACTTCCACTACCACCATGTCACCAAGCTCGACGAGGTGGCTGCC
TCTCAGCTTACCTTCCCTGCTGTCACGCTGTGCAACCTCAACGAGTTCCG
CTTTAGCCAAGTCTCCAAGAATGACCTGTATCATGCTGGCGAGCTGCTGG
CCCTGCTCAACAACAGGTATGAGATACCAGACACACAGATGGCAGATGAA
AAGCAGCTGGAGATACTGCAGGACAAAGCCAACTTCCGCAGCTTCAAACC
CAAACCCTTCAACATGCGCGAGTTCTACGACCGCGCTGGCCACGACATTC
GCGACATGCTGCTCTCCTGCCACTTCCGCGGCGAGGTCTGCAGCGCTGAA
GACTTCAAGGTGGTCTTCACACGCTATGGAAAGTGCTACACGTTCAACTC
CGGCCGCGATGGCCGCCCGCGCCTGAAGACCATGAAGGGTGGCACGGGCA
ATGGCCTGGAAATCATGCTGGACATCCAGCAGGACGAGTACCTGCCTGTG
TGGGGCGAGACTGACGAGACGTCCTTCGAAGCAGGCATCAAAGTGCAGAT
CCATAGTCAGGATGAACCTCCTTTCATCGACCAGCTGGGCTTTGGCGTGG
CCCCAGGCTTCCAGACCTTTGTGGCCTGCCAGGAGCAGCGCCTCATCTAC
CTGCCCCCACCCTGGGGCACCTGCAAAGCTGTTACCATGGACTCCGATTT
GGATTTCTTCGACTCCTACAGCATCACTGCCTGCCGCATCGACTGTGAGA
CGCGCTACCTGGTGGAGAACTGCAACTGCCGCATGGTGCACATGCCAGGC
GATGCCCCATACTGTACTCCAGAGCAGTACAAGGAGTGTGCAGATCCTGC
TCTGGACTTCCTGGTGGAAAAGGACCAGGAGTACTGCGTGTGTGAAATGC
CTTGCAACCTGACCCGCTATGGCAAAGAGCTGTCCATGGTCAAGATCCCC
AGCAAAGCCTCAGCCAAGTACCTGGCCAAGAAGTTCAACAAATCTGAGCA
ATACATAGGCGAGAACATCCTGGTGCTGGACATAACTTTGAAGTCCTCAA
CTATGAGACCATTGAACAGAAGAAGGCCTATGAGATTGCAGGCCTCCTGG
GTGACATCGGCGGCCAGATGGGCCTGTTCATCGGCGCCAGCATCCTCACG
GTGCTGGAGCTCTTTGACTACGCCTACGAGGTCATTAAGCACAAGCTGTG
CCGCCGCGGAAAATGCCAGAAGGAGGCCAAAAGGAGCAGTGCGGACAAGG
GCGTGGCCCTCAGCCTGGACGACGTCAAAAGACACAACCCGTGCGAGAGC
CTTCGCGGCCACCCTGCCGGCATGACATACGCTGCCAACATCCTGCCTCA
CCATCCGGCCCGCGGCACGTTCGAGGACTTTACCTGCTGA 3′),
the forward primer was
(5′ GAAAACCTGTACTTTCAGGGGTCCACACTGCACGGCCTGGCCCACA
3′),
the reverse primer was
(5′ CTTGGTACCGCATGCCTCGAGTTACTTGTGCTTAATGACCTCGTAG
3′),

and the gene ASIC1 (25-464) was obtained by PCR reaction. The forward primer was (5′CCCACCATCGGGCGCGGATCCATGCATCACCATCATCACCACCATCACGA AAACCTGTACTTTCAG 3) the reverse primer was (5′CTTGGTACCGCATGCCTCGAGTTACTTGTGCTTAATGACCTCGTAG 3′), and the gene 8*His-BRIL(23-128)-TEV-ASIC1(26-464) was obtained by PCR reaction. The gene synthesis was carried out according to the above principle, the forward primer was introduced into the restriction enzyme cleavage site BamHI, and the reverse primer was introduced into the restriction enzyme cleavage site XhoI and the stop codon.

The T4L protein (amino acid sequence as set forth in SEQ ID NO.1.2 in the sequence listing) and nucleotide template thereof having a sequence as set forth in SEQ ID NO.2.2 in the sequence listing

(5′ AACATCTTCGAGATGCTCCGTATCGACGAGGGCCTCAGGTTGAAGA
TCTATAAGGACACAGAGGGCTATTACACCATCGGTATTGGTCATCTCTTG
ACTAAGTCGCCTTCACTTAACGCAGCAAAATCCGAACTGGACAAGGCCAT
CGGTCGCAATACAAATGGTGTCATTACTAAGGACGAAGCTGAAAAGCTCT
TCAACCAGGACGTTGATGCAGCTGTTCGCGGAATCCTGCGCAACGCAAAG
CTCAAGCCGGTTTACGATTCTTTGGACGCCGTTCGCCGTGCGGCCTTGAT
TAACATGGTGTTTCAGATGGGTGAAACCGGAGTTGCAGGATTTACCAACT
CGCTGAGGATGCTGCAACAAAAGAGATGGGACGAAGCCGCTGTGAATCTC
GCCAAATCTAGGTGGTACAACCAGACACCCAATCGTGCGAAGAGAGTGAT
AACTACATTCCGCACCGGTACTTGGGACGCATAT 3′),
the forward primer was:
(5′ CCCACCATCGGGCGCGGATCCATGCATCACCATCATCACCACCATC
ACAACATCTTCGAGATGCTCCGT 3′),
the reverse primer was:
(5′ CCCCTGAAAGTACAGGTTTTCATATGCGTCCGTACCGGTG 3′),

and the gene of the T4L protein fragment was obtained by means of PCR reaction.

The ASIC1 nucleotide template was chemically synthesized with reference to human gene codons,

(5′ ATGGAACTGAAGGCCGAGGAGGAGGAGGTGGGTGGCGTCCAGCCGG
TGAGCATCCAGGCCTTCGCCAGCAGCTCCACACTGCACGGCCTGGCCCAC
ATCTTCTCCTACGAGCGCCTGTCTCTGAAGCGCGCACTGTGGGCCCTGTG
CTTCCTGGGCTCCCTGGCTGTGCTGCTGTGTGTGTGCACGGAGCGCGTGC
AGTACTACTTCCACTACCACCATGTCACCAAGCTCGACGAGGTGGCTGCC
TCTCAGCTTACCTTCCCTGCTGTCACGCTGTGCAACCTCAACGAGTTCCG
CTTTAGCCAAGTCTCCAAGAATGACCTGTATCATGCTGGCGAGCTGCTGG
CCCTGCTCAACAACAGGTATGAGATACCAGACACACAGATGGCAGATGAA
AAGCAGCTGGAGATACTGCAGGACAAAGCCAACTTCCGCAGCTTCAAACC
CAAACCCTTCAACATGCGCGAGTTCTACGACCGCGCTGGCCACGACATTC
GCGACATGCTGCTCTCCTGCCACTTCCGCGGCGAGGTCTGCAGCGCTGAA
GACTTCAAGGTGGTCTTCACACGCTATGGAAAGTGCTACACGTTCAACTC
CGGCCGCGATGGCCGCCCGCGCCTGAAGACCATGAAGGGTGGCACGGGCA
ATGGCCTGGAAATCATGCTGGACATCCAGCAGGACGAGTACCTGCCTGTG
TGGGGCGAGACTGACGAGACGTCCTTCGAAGCAGGCATCAAAGTGCAGAT
CCATAGTCAGGATGAACCTCCTTTCATCGACCAGCTGGGCTTTGGCGTGG
CCCCAGGCTTCCAGACCTTTGTGGCCTGCCAGGAGCAGCGCCTCATCTAC
CTGCCCCCACCCTGGGGCACCTGCAAAGCTGTTACCATGGACTCCGATTT
GGATTTCTTCGACTCCTACAGCATCACTGCCTGCCGCATCGACTGTGAGA
CGCGCTACCTGGTGGAGAACTGCAACTGCCGCATGGTGCACATGCCAGGC
GATGCCCCATACTGTACTCCAGAGCAGTACAAGGAGTGTGCAGATCCTGC
TCTGGACTTCCTGGTGGAAAAGGACCAGGAGTACTGCGTGTGTGAAATGC
CTTGCAACCTGACCCGCTATGGCAAAGAGCTGTCCATGGTCAAGATCCCC
AGCAAAGCCTCAGCCAAGTACCTGGCCAAGAAGTTCAACAAATCTGAGCA
ATACATAGGCGAGAACATCCTGGTGCTGGACATTTTCTTTGAAGTCCTCA
ACTATGAGACCATTGAACAGAAGAAGGCCTATGAGATTGCAGGCCTCCTG
GGTGACATCGGCGGCCAGATGGGCCTGTTCATCGGCGCCAGCATCCTCAC
GGTGCTGGAGCTCTTTGACTACGCCTACGAGGTCATTAAGCACAAGCTGT
GCCGCCGCGGAAAATGCCAGAAGGAGGCCAAAAGGAGCAGTGCGGACAAG
GGCGTGGCCCTCAGCCTGGACGACGTCAAAAGACACAACCCGTGCGAGAG
CCTTCGCGGCCACCCTGCCGGCATGACATACGCTGCCAACATCCTGCCTC
ACCATCCGGCCCGCGGCACGTTCGAGGACTTTACCTGCTGA 3′),
the forward primer was
(5′ GAAACCTGTTACTTTCAGGGGTCCACACTGCACGGCCTGGCCCACA
3′),
the reverse primer was
(5′ CTTGGTACCGCATGCCTCGAGTTACTTGTGCTTAATGACCTCGTAG
3′),

and the gene ASIC1 (25-464) was obtained by PCR reaction. The forward primer was (5′CCCACCATCGGGCGCGGATCCATGCATCACCATCATCACCACCATCACGA AAACCTGTACTTTCAG 3′), the reverse primer was (5′CTTGGTACCGCATGCCTCGAGTTACTTGTGCTTAATGACCTCGTAG 3′), and the gene 8*His-T4L(2-161)-TEV-ASIC1(25-464) was obtained by PCR reaction. The gene was synthesized according to the above principle, the forward primer was introduced into the restriction enzyme cleverage site BamHI, and the reverse primer was introduced into the restriction enzyme cleverage site XhoI and the stop codon.

The PCR reaction conditions were as lows: 0.2 μM of an amplification primer was added to a 50 μL of reaction system (PCR Buffer, 1.5 mM MgSO₄, 200 μM dNTPs), and PCR cycle was started after a thorough mixing: denaturation at 94° C. for 5 minutes, denaturation at 94° C. for 30 seconds, annealing at 55° C. for 30 seconds, and elongation at 68° C. for 2 minutes; total 30 cycles were carried out, and finally the temperature was kept at 68° C. for 10 minutes. The PCR products were identified by 1.2% agarose gel electrophoresis, and recovered for cloning.

### EXAMPLE 2

### Construction of Plasmid for an ASIC1 (Acid-Sensing Ion Channel 1) Fusion Protein

The PCR fragment obtained in Example 1 was ligated with the vector pFastBac1 (purchased from Invitrogen) double-cleaved with the two restriction enzymes EcoRI and XhoI. The ligated product was transformed into the competent E. coli (Escherichia coli) DH5α and the volume thereof should not exceed 10% of the competent cells. The contents were homogenously mixed by gently rotating for several times, the tubes with contents were cooled in an ice bath for 30 minutes, and the tubes were placed in a 42° C. water bath and subjected to thermal shock for 60 seconds. The tubes were quickly transferred to an ice bath and stood for 120 seconds so as to cool the cells. 400 μl of LB culture medium was added to each tube and and the tube was incubated at 37° C. for 60 minutes under slowly shaking so that the bacteria were resuscitated to express a plasmid-encoded antibiotic resistance marker gene was expressed, and centrifuged at low speed for 2 minutes. The supernatant was removed, leaving about 100 μl of culture medium in the centrifuge tube, and re-suspending bacteria, and the solution of bacteria was homogenously spread with a glass spatula on an agar plate. The plate was inverted in a constant temperature incubator at 37° C., and colonies might emerge after 12-16 hours. The positive clones were picked and identified after application on the plate.

### EXAMPLE 3

### Expression of the ASIC1 (Acid-Sensing Ion Channel 1) Fusion Protein

The recombinant plasmid obtained in Example 2 was transformed into DH10Bac E. coli competent cells, and the volume thereof should not exceed 5% of the competent cells. The contents were homogenously mixed by gently rotating, for several times. The tubes with contents were cooled in an ice bath for 30 minutes, and the tubes were placed in a 42° C. water bath and subjected to thermal shock for 90 seconds. The tubes were quickly transferred, to an ice bath and stood for 120 seconds so as to cool the cells. 800 μl of LB culture medium was added to each tube and the tube was incubated at 37° C. for 4 hours under slowly shaking so that the bacteria were resuscitated to express a plasmid-encoded antibiotic resistance marker gene. 30 μl of the solution of bacteria was homogenously spread with a glass spatula on an agar plate. The plate was inverted in a constant temperature incubator at 37° C., and positive blue and white spot colonies emerged after 30-48 hours, The positive white spot colonies were picked into 5 ml of resistant LB, and incubated for 12-16 hours under slowly shaking, and the bacteria were identified by PCR. The results showed that bacmid recombination was correct. The recombinant bacmid was transfected into insect cells with a transfection reagent. After 4-5 days, the cell supernatant was collected as the first generation baculovirus. The second generation virus for expressing the fusion polypeptide was obtained after 72 hours of infection of the insect cells with the first generation baculovirus. The insect cells sf9 with a density of 2×10⁶/ml were infected by the second generation virus at a volume ratio of 1:100. After 72 hours of further incubation, cells were harvested by centrifugation and washed with PBS for once.

### EXAMPLE 4

### Purification of the ASIC1 (Acid-Sensing Ion Channel 1) Fusion Protein

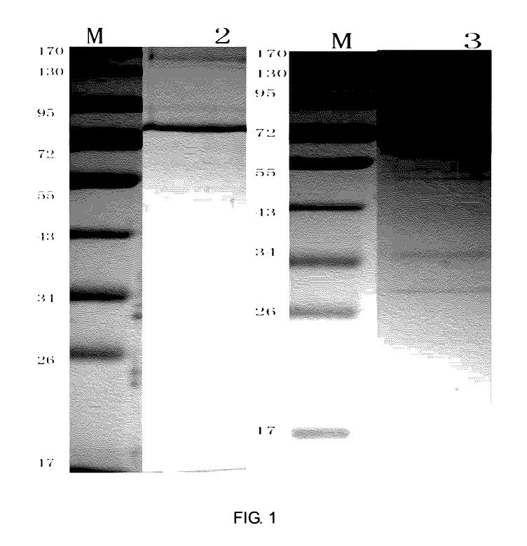

1 L of cells were re-suspended in 100 ml of a precooled lysate (25 mM Tris pH 8.0 10 mM MgCl₂, 20 mM KCl), homogenized on ice using a homogenizer, and centrifuged with an ultracentrifuge for 45 minutes after homogenization; the supernatant was removed; the washing step was repeated for three times, and then the washing step was repeated with a high-salt solution for three times. The extracted cell membrane was dissolved with a lysis solution containing glycerol, quickly frozen with liquid nitrogen, and stored in a ˜80° C. refrigerator, The cell membrane was thawed and defrosted on ice. After 30 minutes, a buffer for dissolving membranes was added to the cell me b in an amount of 100 ml per 1 L of the cell membrane, and then the dissolved membranes were placed on ice overnight, and centrifuged in, an ultracentrifuge at 100,000 g of of centrifugal force for 1 hour. The precipitate was removed, and the supernatant was then incubated overnight with 1 mL of Talon IMAC resin balanced with a balance buffer. In the next day, the supernatant was removed, and an appropriate amount of balance buffer was added to re-suspend packing. The packing was transferred to a gravity column, and washed with 10 column volumes of a rinse buffer 1 (25 mM Tris pH8.0; 150 mM NaCl, 0.05% DDM; 5 mM MgCl₂), 10 column volumes of a rinse buffer 2 (25 mM Tris pH8.0; 150 mM NaCl, 0.05% DDM; 5 mM MgCl₂; 25 mM Imid), and 5 column volumes of a rinse buffer 3 (25 mM Tris pH8.0; 150 mM NaCl; 0.05% DDM; 5 mM MgCl; 50 mM Imid), and then the proteins of interest were eluted with 5 column volumes of elution buffer (25 mM Tris pH8.0; 150 mM NaCl; 0.05% DDM; 5 mM MgCl₂; 250 mM Imid). The purified proteins of interest were stored at ˜80° C. The results of electrophoresis of the three fusion proteins were shown in FIG. 1.

As viewed from yield, the yield of the three purified membrane proteins exceeded 1 mg/L cells, suggesting a high yield.

### EXAMPLE 5

### Detection of Homogeneity of the Inventive ASIC1 (Acid-Sensing Ion Channel 1) Fusion Protein

The detection was carried out by using Acquity H-Class Bio UPLC system from Wasters Company, wherein the column used in the detection was a Sepax EC250 molecular sieve column. Prior to sample application, the column was washed with a balance buffer (25 mM Tris pH 5.0; 150 mM NaCl; 0.05% DDM; 5 mM MgCl₂) until the baseline was not much changed, and then a sample to be detected was added into a special 96-hole plate. The integration treatment was performed by softwares of the instrument per se.

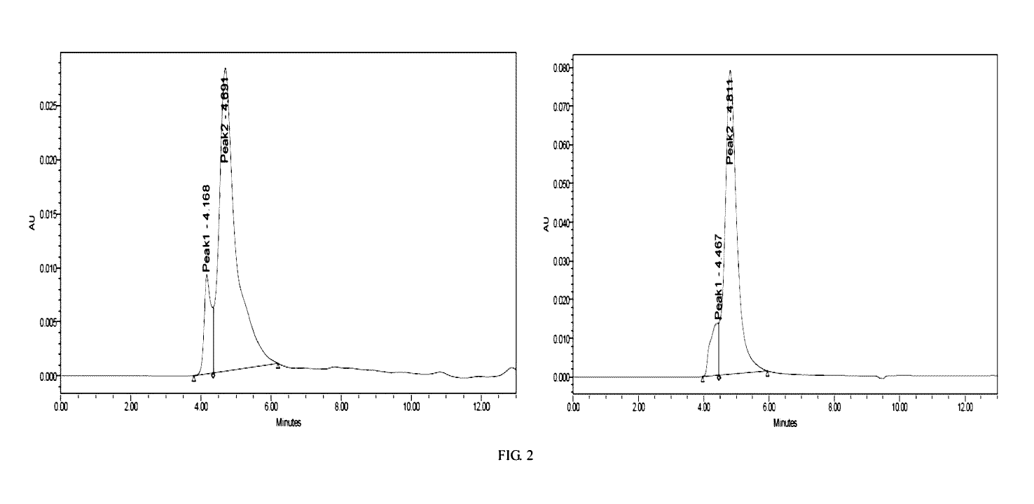

The three fusion proteins were detected according to the above-described method and the results were shown in FIG. 2. The results showed that the three fusion proteins have good homogeneity, a monomer peak of the fusion proteins is detectable, and the monomer accounts for a majority of the protein sample.

### EXAMPLE 6

### Synthesis of Gene of GLUT1 (Glucose Transporter) Fusion Protein

The T4L protein (amino acid sequence as set forth in SEQ ID NO.1.2 in the sequence listing) and nucleotide template thereof having a sequence as set forth in SEQ ID NO.2.2 in the sequence listing

(5′ AACATCTTCGAGATGCTCCGTATCGACGAGGGCCTCAGGTTGAAGA
TCTATAAGGACACAGAGGGCTATTACACCATCGGTATTGGTCATCTCTTG
ACTAAGTCGCCTTCACTTAACGCAGCAAAATCCGAACTGGACAAGGCCAT
CGGTCGCAATACAAATGGTGTCATTACTAAGGACGAAGCTGAAAAGCTCT
TCAACCAGGACGTTGATGCAGCTGTTCGCGGAATCCTGCGCAACGCAAAG
CTCAAGCCGGTTTACGATTCTTTGGACGCCGTTCGCCGTGCGGCCTTGAT
TAACATGGTGTTTCAGATGGGTGAAACCGGAGTTGCAGGATTTACCAACT
CGCTGAGGATGCTGCAACAAAAGAGATGGGACGAAGCCGCTGTGAATCTC
GCCAAATCTAGGTGGTACAACCAGACACCCAATCGTGCGAAGAGAGTGAT
AACTACATTCCGCACCGGTACTTGGGACGCATAT 3′),
the forward primer was:
(5′ CCCGTTTCTGCTAGCAAGCTTACATGATCTCGAGATGCTCCGTATC
3′),
the reverse primer was:
(5′ CAGCTTCTTGCTGCTGGGCTCCATATATGGTCCCAAGTACCGGTGC
3′),

and the gene of the T4L protein fragment was obtained by PCR reaction.

The GLUT1 nucleotide template was chemically synthesized with reference to human gene codons.

(5′ ATGGAGCCCAGCAGCAAGAAGCTGACGGGTCGCCTCATGCTGGCCG
TGGGAGGAGCAGTGCTTGGCTCCCTGCAGTTTGGCTACAACACTGGAGTC
ATCAATGCCCCCCAGAAGGTGATCGAGGAGTTCTACAACCAGACATGGGT
CCACCGCTATGGGGAGAGCATCCTGCCCACCACGCTCACCACGCTCTGGT
CCCTCTCAGTGGCCATCTTTCTGTTGGGGGCATGATTGGCTCCTTCTCTG
TGGGCCTTTTCGTTAACCGCTTTGGCCGGCGGAATTCAATGCTGATGATG
AACCTGCTGGCCTTCGTGTCCGCCGTGCTCATGGGCTTCTCGAAACTGGG
CAAGTCCTTTGAGATGCTGATCCTGGGCCGCTTCATCATCGGTGTGTACT
GCGGCCTGACCACAGGCTTCGTGCCCATGTATGTGGGTGAAGTGTCACCC
ACAGCCCTTCGTGGGGCCCTGGGCACCCTGCACCAGCTGGGCATCGTCGT
CGGCATCCTCATCGCCCAGGTGTTCGGCCTGGACTCCATCATGGGCAACA
AGGACCTGTGGCCCCTGCTGCTGAGCATCATCTTCATCCCGGCCCTGCTG
CAGTGCATCGTGCTGCCCTTCTGCCCCGAGAGTCCCCGCTTCCTGCTCAT
CAACCGCAACGAGGAGAACCGGGCCAAGAGTGTGCTAAAGAAGCTGCGCG
GGACAGCTGACGTGACCCATGACCTGCAGGAGATGAAGGAAGAGAGTCGG
CAGATGATGCGGGAGAAGAAGGTCACCATCCTGGAGCTGTTCCGCTCCCC
CGCCTACCGCCAGCCCATCCTCATCGCTGTGGTGCTGCAGCTGTCCCAGC
AGCTGTCTGGCATCAACGCTGTCTTCTATTACTCCACGAGCATCTTCGAG
AAGGCGGGGGTGCAGCAGCCTGTGTATGCCACCATTGGCTCCGGTATCGT
CAACACGGCCTTCACTGTCGTGTCGCTGTTTGTGGTGGAGCGAGCAGGCC
GGCGGACCCTGCACCTCATAGGCCTCGCTGGCATGGCGGGTTGTGCCATA
CTCATGACCATCGCGCTAGCACTGCTGGAGCAGCTACCCTGGATGTCCTA
TCTGAGCATCGTGGCCATCTTTGGCTTTGTGGCCTTCTTTGAAGTGGGTC
CTGGCCCCATCCCATGGTTCATCGTGGCTGAACTCTTCAGCCAGGGTCCA
CGTCCAGCTGCCATTGCCGTTGCAGGCTTCTCCAACTGGACCTCAAATTT
CATTGTGGGCATGTGCTTCCAGTATGTGGAGCAACTGTGTGGTCCCTACG
TCTTCATCATCTTCACTGTGCTCCTGGTTCTGTTCTTCATCTTCACCTAC
TTCAAAGTTCCTGAGACTAAAGGCCGGACCTTCGATGAGATCGCTTCCGG
CTTCCGGCAGGGGGGAGCCAGCCAAAGTGACAAGACACCCGAGGAGCTGT
TCCATCCCCTGGGGGCTGATTCCCAAGTGTGA 3′),
the forward primer was
(5′ ATGGAGCCCAGCAGCAAGAAGCTG 3′),
the reverse primer was
(5′ ATGATGGTGGTGATGGTGGTGATGGTGGTGTGTCTTGTCACTTTGG
CTGGCTC 3′),

and the gene GLUT1(1-478) was obtained by PCR reaction. The forward primer was (5′CCCGTTTCTGCTAGCAAGCTTACCATGAACATCTTCGAGATGCTCCGTATC 3), the reverse primer was (5′GGTCGAGGTCGGGGGATCCTTAATGATGGTGGTGATGGTGGTGATG 3), and the gene T4L-GLUT1(1-478)-10*His was obtained by means of PCR reaction. The gene was synthesized according to the above principle, the forward primer was introduced into the restriction enzyme cleverage site HindIII, and the reverse primer was introduced into the restriction enzyme cleverage site BamHI and the stop codon.

BRIL protein fragment (amino acid sequence as set forth in SEQ ID NO.1.1), nucleotide template (SEQ ID NO.2.1)

5′ GCTGATCTGGAAGACAATTGGGAAACTCTGAACGACAATCTCAAGGT
GATCGAGAAGGCTGACAATGCTGCACAAGTCAAAGACGCTCTGACCAAGA
TGAGGGCAGCAGCCCTGGACGCTCAGAAGGCCACTCCACCTAAGCTCGAG
GACAAGAGCCCAGATAGCCCTGAAATGAAAGACTTTCGGCATGGATTCGA
CATTCTGGTGGGACAGATTGATGATGCACTCAAGCTGGCCAATGAAGGGA
AAGTCAAGGAAGCACAAGCAGCCGCTGAGCAGCTGAAGACCACCCGGAAT
GCATACATTCAGAAGTACCTG 3′),
the forward primer was:
(5′ GCTGATCTGGAAGACAATTGGGAAAC 3′),
the reverse primer was:
(5′ CAGGTACTTCTGAATGTATGCATTC′),

and the gene of the BRIL protein fragment was obtained by means of PCR reaction.

The GLUT1 nucleotide template was chemically synthesized with reference to human gene codons,

(5′ ATGGAGCCCAGCAGCAAGAAGCTGACGGGTCGCCTCATGCTGGCCG
TGGGAGGAGCAGTGCTTGGCTCCCTGCAGTTTGGCTACAACACTGGAGTC
ATCAATGCCCCCCAGAAGGTGATCGAGGAGTTCTACAACCAGACATGGGT
CCACCGCTATGGGGAGAGCATCCTGCCCACCACGCTCACCACGCTCTGGT
CCCTCTCAGTGGCCATCTTTCTGTTGGGGGCATGATTGGCTCCTTCTCTG
TGGGCCTTTTCGTTAACCGCTTTGGCCGGCGGAATTCAATGCTGATGATG
AACCTGCTGGCCTTCGTGTCCGCCGTGCTCATGGGCTTCTCGAAACTGGG
CAAGTCCTTTGAGATGCTGATCCTGGGCCGCTTCATCATCGGTGTGTACT
GCGGCCTGACCACAGGCTTCGTGCCCATGTATGTGGGTGAAGTGTCACCC
ACAGCCCTTCGTGGGGCCCTGGGCACCCTGCACCAGCTGGGCATCGTCGT
CGGCATCCTCATCGCCCAGGTGTTCGGCCTGGACTCCATCATGGGCAACA
AGGACCTGTGGCCCCTGCTGCTGAGCATCATCTTCATCCCGGCCCTGCTG
CAGTGCATCGTGCTGCCCTTCTGCCCCGAGAGTCCCCGCTTCCTGCTCAT
CAACCGCAACGAGGAGAACCGGGCCAAGAGTGTGCTAAAGAAGCTGCGCG
GGACAGCTGACGTGACCCATGACCTGCAGGAGATGAAGGAAGAGAGTCGG
CAGATGATGCGGGAGAAGAAGGTCACCATCCTGGAGCTGTTCCGCTCCCC
CGCCTACCGCCAGCCCATCCTCATCGCTGTGGTGCTGCAGCTGTCCCAGC
AGCTGTCTGGCATCAACGCTGTCTTCTATTACTCCACGAGCATCTTCGAG
AAGGCGGGGGTGCAGCAGCCTGTGTATGCCACCATTGGCTCCGGTATCGT
CAACACGGCCTTCACTGTCGTGTCGCTGTTTGTGGTGGAGCGAGCAGGCC
GGCGGACCCTGCACCTCATAGGCCTCGCTGGCATGGCGGGTTGTGCCATA
CTCATGACCATCGCGCTAGCACTGCTGGAGCAGCTACCCTGGATGTCCTA
TCTGAGCATCGTGGCCATCTTTGGCTTTGTGGCCTTCTTTGAAGTGGGTC
CTGGCCCCATCCCATGGTTCATCGTGGCTGAACTCTTCAGCCAGGGTCCA
CGTCCAGCTGCCATTGCCGTTGCAGGCTTCTCCAACTGGACCTCAAATTT
CATTGTGGGCATGTGCTTCCAGTATGTGGAGCAACTGTGTGGTCCCTACG
TCTTCATCATCTTCACTGTGCTCCTGGTTCTGTTCTTCATCTTCACCTAC
TTCAAAGTTCCTGAGACTAAAGGCCGGACCTTCGATGAGATCGCTTCCGG
CTTCCGGCAGGGGGGAGCCAGCCAAAGTGACAAGACACCCGAGGAGCTGT
TCCATCCCCTGGGGGCTGATTCCCAAGTGTGA 3′),
the forward primer was
(5′ CCCGTTCTGCTAGCAAGCTTACCATGGAGCCCAGCAGCAAGAAGCT
G 3′),
the reverse primer was
(5′ GTTTCCCAATTGTCTTCCAGATCAGCCTTGGCCCGGTTCTCCTCGT
TG 3′)

and the gene GLUT1(1-225) was obtained by PCR reaction; the forward primer was (5′GAATGCATACATTCAGAAGTACCTGGAGAAGAAGGTCACCATCCTGGAGC 3′), the reverse primer was (5′ATGATGGTGGTGATGGTGGTGATGGTGGTGCACTTGGGAATCAGGCCCC A 3′), and the gene GLUT1(254-492) was obtained by PCR reaction. The forward primer was (5′CCCGTTTCTGCTAGCAAGCTTACCATGGAGCCCAGCAGCAAGAAGCTG 3′), the reverse primer was (5′GGTCGAGGTCGGGGGATCCTTAATGATGGTGGTGATGGTGGIGATG and the gene GLUT1(1-225)-BRIL(130-233)-TEV-GLUT1(254-492)-10*His was obtained by PCR reaction. The gene was synthesized according to the above principle, the forward primer was introduced into the restriction enzyme cleverage site Hind III, and the reverse primer was introduced into the restriction enzyme cleverage site BamHI and the stop codon.

### EXAMPLE 7

### Expression of the Inventive GLUT1 (Glucose Transporter) Fusion Protein

1 mg of the GLUT1 plasmid in Example 6 was dissolved in 50 ml of Opti-MEM culture medium and homogenously mixed, then further dissolved with 3 ml of PEI (1 mg/ml) in 50 Opti-MEM culture medium and mixed homogenously, incubated at room temperature for 15 minutes, added to 1 L of 293F cells with a cell density of 1.2×10⁶ cells/ml, and cultured at 37° C. in 5% CO₂ for 72 hours, and the cell precipitates were collected.

### EXAMPLE 8

### Purification of the Inventive GLUT1 (Glucose Transporter) Fusion Protein

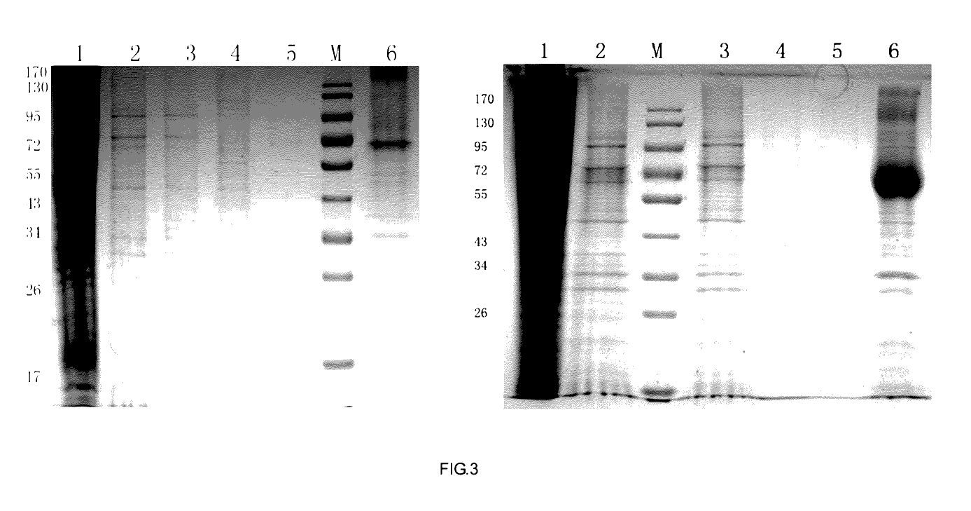

300 mL of cells were re-suspended in 20 ml of a precooled lysate (50 mM Tris pH7.5, 200 mM NaCl, 5% glycerol), homogenized on ice using a homogenizer, and centrifuged with an ultracentrifuge for 50 minutes after homogenization; the supernatant was removed; the washing step was repeated for three times, and then the washing step was repeated with a high-salt solution (50 mM Tris pH7.5, 1M NaCl, 5% glycerol) for twice. A buffer for dissolving membranes (50 mM Tris pH7.5, 200 mM NaCl, 5% glycerol, 1% DDM) was added to the extracted cell membrane in an amount of 20 ml per 300 mL of cells, homogenized on ice using a homogenizer, the dissolved membranes were stirred using a magnetic stirring apparatus at 4° C. overnight, and centrifuged in an ultracentrifuge at 100,000 g of centrifugal force for 1 hour. The precipitate was removed, and the supernatant was then incubated with 1 mL of Talon IMAC resin balanced with a balance buffer (50 Mm Tris pH7.5, 200 mM NaCl, 5% glycerol, 0.05% DDM) for 3 hours. The supernatant was removed, and an appropriate amount of balance buffer was added to re-suspend packing. The packing was transferred to a gravity column, arid washed with 10 column volumes of a rinse buffer 1 (250 mil Tris pH7.5, 200 nM NaCl, 5% glycerol, 0.05% DDM), 1 column volumes of a rinse buffer 2 (50 mM Tris pH7.5, 200 mM NaCl, 5% glycerol, 0.05% DDM, 20 mM imidazole), and 5 column volumes of a rinse buffer 3 (50 mM Tris pH7.5, 200 mM NaCl, 5% glycerol, 0.05% DDM, 50 mM imidazole), and then the proteins of interest were eluted with 5 column volumes of elution buffer (50 mM Tris pH7.5, 200 mM NaCl, 5% glycerol, 0.05% DDM, 250 mM imidazole). The purified proteins of interest were concentrated to 400 μL and stored at ˜80° C. The results of electrophoresis of the three fusion proteins were shown in FIG. 3.

As viewed from yield, the yield of the two purified GLUT1 fusion proteins exceeded 0.5 mg/L cells, suggesting a high yield.

### EXAMPLE 9

### Detection of Homogeneity of the Inventive GLUT1 (Glucose Transporter) Fusion Protein

The detection was carried out by using Acquity H-Class Bio UPLC system from Wasters Company, wherein the column used in the assay was a Sepax SEC250 molecular sieve column_{—} Prior to sample application, the column was washed with a balance buffer (25 mM Tris pH 8.0; 150 mM NaCl; 0.05% DDM; 5 mM MgCl₂) until the baseline was not much changed, and then a sample to detected was added into a special 96-hole plate. The integration treatment was performed by softwares of the instrument per se.

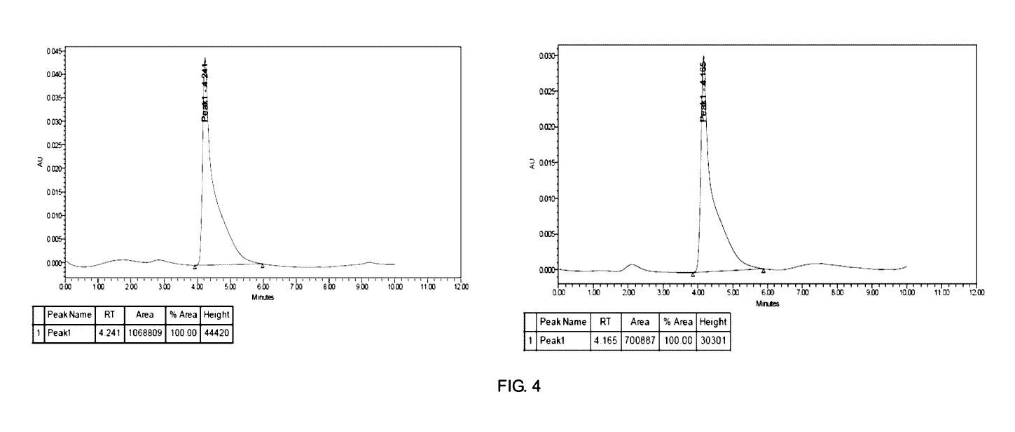

The three fusion proteins were detected according to the above-described method, and the results were shown in FIG. 4. The results showed that the two GLUT1 fusion proteins have good homogeneity, a monomer peak of the fusion proteins is detectable, and the monomer accounts for a majority of the protein sample.

We claim:

1. A protein fragment for fusion expression of ion channel protein and transport protein, characterized in that the protein fragment is a Bril protein fragment or a protein having a sequence homology of greater than 90% with this fragment, or a T4L protein fragment or a protein having a sequence homology of greater than 90% with this fragment.

2. The protein fragment for fusion expression of ion channel protein and transport protein according to claim 1, characterized in that said Bril protein fragment or the protein having a sequence homology of greater than 90% with this fragment has an amino acid sequence as set forth in SEQ ID NO.1.1 and a coding gene sequence as set forth in SEQ ID NO.2.1; and the said T4L protein fragment or the protein having a sequence homology of greater than 90% with this fragment has an amino acid sequence as set forth in SEQ ID NO.1.2 and a coding gene sequence as set forth in SEQ ID NO.2.2.

3. A method for fusion expression of ion channel protein and transport protein according to claim 1, characterized in that the amino acid sequence of the protein fragment as defined in claim 1 is inserted into the N-terminal, C-terminal or intramembrane loop region of the ion channel protein or transport protein to construct a fusion protein.

4. The method for fusion expression of ion channel protein and transport protein according to claim 3, characterized in that said Bril protein fragment and an ASIC1 are subjected to fusion expression to obtain a fusion protein having an amino acid sequence as set forth in SEQ ID NO.3.1; or said T4L protein fragment and an ASIC1 are subjected to fusion expression to obtain a fusion protein having an amino acid sequence as set forth in SEQ ID NO.3.2.

5. The method for fusion expression of ion channel protein and, transport protein according to claim 3, characterized in that an ASIC1 fusion expression plasmid constructed with the Bril gene has a gene sequence as set forth in SEQ ID NO.4.1; or an ASIC1 fusion expression plasmid constructed with the T4L gene has a gene sequence as set forth in SEQ ID NO.4.2.

6. The method for fusion expression of ion channel protein and transport protein according to claim 3, characterized in that said Bril protein fragment and a GLUT1 are subjected to fusion expression to obtain a fusion protein having an amino acid sequence as set forth in SEQ ID NO.3.3; or said T4L protein fragment and a GLUT1 are subjected to fusion expression to obtain a fusion protein having an amino acid sequence as set forth in SEQ ID NO.3.4.

7. The method for fusion expression of ion channel protein and transport protein according to claim 3, characterized in that a GLUT1 fusion expression plasmid constructed with the Bril gene has a gene sequence as set forth in SEQ ID NO.4.3; or a GLUT1 fusion expression plasmid constructed with the T4L, gene has a gene sequence as set forth in SEQ ID NO.4.4.

8. The fusion expression plasmid according to claim 5, characterized in that the fusion expression plasmid can be expressed in insect cells or can be expressed in mammalian cells.

9. The method for fusion expression of ion channel protein and transport protein according to claim 5, characterized in that the gene sequence as set forth in SEQ ID NO.4.1 or SEQ ID NO.4.2 described in claim 5 is constructed into an expression vector, such as pFastBac1, PcDNA3.1, PET21b and the like, for protein expression in different expression systems.

10. The method for fusion expression of ion channel protein and transport protein according to claim 7, characterized in that the gene sequence as set forth in SEQ ID NO.4.3 or SEQ ID NO.4.4 described in claim 7 is constructed into an expression vector, such as pFastBac1, PcDNA3.1, PET21b and the like, for protein expression in different expression systems.

11. The fusion expression plasmid according to claim 7, characterized in that the fusion expression plasmid can be expressed in insect cells or can be expressed in mammalian cells.


SEQUENCE LISTING (38 sequences)

SEQ ID NO: 1 - protein, 106 aa, Unknown
  BRIL protein
ADLEDNWETLNDNLKVIEKADNAAQVKDALTKMRAAALDAQKATPPKLEDKSPDSPEMKDFRHGFDILVGQIDDALKLANEGKVKEAQAAAEQLKTTRNAYIQKYL

SEQ ID NO: 2 - protein, 160 aa, Unknown
  T4L protein
NIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAY

SEQ ID NO: 3 - DNA, 318 bp, Unknown
  BRIL protein
gctgatctggaagacaattgggaaactctgaacgacaatctcaaggtgatcgagaaggctgacaatgctgcacaagtcaaagacgctctgaccaagatgagggcagcagccctggacgctcagaaggccactccacctaagctcgaggacaagagcccagatagccctgaaatgaaagactttcggcatggattcgacattctggtgggacagattgatgatgcactcaagctggccaatgaagggaaagtcaaggaagcacaagcagccgctgagcagctgaagaccacccggaatgcatacattcagaagtacctg

SEQ ID NO: 4 - DNA, 480 bp, Unknown
  T4L protein
aacatcttcgagatgctccgtatcgacgagggcctcaggttgaagatctataaggacacagagggctattacaccatcggtattggtcatctcttgactaagtcgccttcacttaacgcagcaaaatccgaactggacaaggccatcggtcgcaatacaaatggtgtcattactaaggacgaagctgaaaagctcttcaaccaggacgttgatgcagctgttcgcggaatcctgcgcaacgcaaagctcaagccggtttacgattctttggacgccgttcgccgtgcggccttgattaacatggtgtttcagatgggtgaaaccggagttgcaggatttaccaactcgctgaggatgctgcaacaaaagagatgggacgaagccgctgtgaatctcgccaaatctaggtggtacaaccagacacccaatcgtgcgaagagagtgataactacattccgcaccggtacttgggacgcatat

SEQ ID NO: 5 - protein, 562 aa, Unknown
  Fusion expression of ASIC1 protein and BRIL protein
MHHHHHHHHADLEDNWETLNDNLKVIEKADNAAQVKDALTKMRAAALDAQKATPPKLEDKSPDSPEMKDFRHGFDILVGQIDDALKLANEGKVKEAQAAAEQLKTTRNAYIQKYLENLYFQGSTLHGLAHIFSYERLSLKRALWALCFLGSLAVLLCVCTERVQYYFHYHHVTKLDEVAASQLTFPAVTLCNLNEFRFSQVSKNDLYHAGELLALLNNRYEIPDTQMADEKQLEILQDKANFRSFKPKPFNMREFYDRAGHDIRDMLLSCHFRGEVCSAEDFKVVFTRYGKCYTFNSGRDGRPRLKTMKGGTGNGLEIMLDIQQDEYLPVWGETDETSFEAGIKVQIHSQDEPPFIDQLGFGVAPGFQTFVACQEQRLIYLPPPWGTCKAVTMDSDLDFFDSYSITACRIDCETRYLVENCNCRMVHMPGDAPYCTPEQYKECADPALDFLVEKDQEYCVCEMPCNLTRYGKELSMVKIPSKASAKYLAKKFNKSEQYIGENILVLDIFFEVLNYETIEQKKAYEIAGLLGDIGGQMGLFIGASILTVLELFDYAYEVIKHK

SEQ ID NO: 6 - protein, 616 aa, Unknown
  Fusion expression of ASIC1 protein and T4L protein
MHHHHHHHHNIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAYENLYFQGSTLHGLAHIFSYERLSLKRALWALCFLGSLAVLLCVCTERVQYYFHYHHVTKLDEVAASQLTFPAVTLCNLNEFRFSQVSKNDLYHAGELLALLNNRYEIPDTQMADEKQLEILQDKANFRSFKPKPFNMREFYDRAGHDIRDMLLSCHFRGEVCSAEDFKVVFTRYGKCYTFNSGRDGRPRLKTMKGGTGNGLEIMLDIQQDEYLPVWGETDETSFEAGIKVQIHSQDEPPFIDQLGFGVAPGFQTFVACQEQRLIYLPPPWGTCKAVTMDSDLDFFDSYSITACRIDCETRYLVENCNCRMVHMPGDAPYCTPEQYKECADPALDFLVEKDQEYCVCEMPCNLTRYGKELSMVKIPSKASAKYLAKKFNKSEQYIGENILVLDIFFEVLNYETIEQKKAYEIAGLLGDIGGQMGLFIGASILTVLELFDYAYEVIKHK

SEQ ID NO: 7 - DNA, 1689 bp, Unknown
  ASIC1 protein and BRIL fusion protein
atgcatcaccatcatcaccaccatcacgctgatctggaagacaattgggaaactctgaacgacaatctcaaggtgatcgagaaggctgacaatgctgcacaagtcaaagacgctctgaccaagatgagggcagcagccctggacgctcagaaggccactccacctaagctcgaggacaagagcccagatagccctgaaatgaaagactttcggcatggattcgacattctggtgggacagattgatgatgcactcaagctggccaatgaagggaaagtcaaggaagcacaagcagccgctgagcagctgaagaccacccggaatgcatacattcagaagtacctggaaaacctgtactttcaggggtccacactgcacggcctggcccacatcttctcctacgagcgcctgtctctgaagcgcgcactgtgggccctgtgcttcctgggctccctggctgtgctgctgtgtgtgtgcacggagcgcgtgcagtactacttccactaccaccatgtcaccaagctcgacgaggtggctgcctctcagcttaccttccctgctgtcacgctgtgcaacctcaacgagttccgctttagccaagtctccaagaatgacctgtatcatgctggcgagctgctggccctgctcaacaacaggtatgagataccagacacacagatggcagatgaaaagcagctggagatactgcaggacaaagccaacttccgcagcttcaaacccaaacccttcaacatgcgcgagttctacgaccgcgctggccacgacattcgcgacatgctgctctcctgccacttccgcggcgaggtctgcagcgctgaagacttcaaggtggtcttcacacgctatggaaagtgctacacgttcaactccggccgcgatggccgcccgcgcctgaagaccatgaagggtggcacgggcaatggcctggaaatcatgctggacatccagcaggacgagtacctgcctgtgtggggcgagactgacgagacgtccttcgaagcaggcatcaaagtgcagatccatagtcaggatgaacctcctttcatcgaccagctgggctttggcgtggccccaggcttccagacctttgtggcctgccaggagcagcgcctcatctacctgcccccaccctggggcacctgcaaagctgttaccatggactccgatttggatttcttcgactcctacagcatcactgcctgccgcatcgactgtgagacgcgctacctggtggagaactgcaactgccgcatggtgcacatgccaggcgatgccccatactgtactccagagcagtacaaggagtgtgcagatcctgctctggacttcctggtggaaaaggaccaggagtactgcgtgtgtgaaatgccttgcaacctgacccgctatggcaaagagctgtccatggtcaagatccccagcaaagcctcagccaagtacctggccaagaagttcaacaaatctgagcaatacataggcgagaacatcctggtgctggacattttctttgaagtcctcaactatgagaccattgaacagaagaaggcctatgagattgcaggcctcctgggtgacatcggcggccagatgggcctgttcatcggcgccagcatcctcacggtgctggagctctttgactacgcctacgaggtcattaagcacaagtaa

SEQ ID NO: 8 - DNA, 1851 bp, Unknown
  ASIC1 protein and T4L fuison protein
atgcatcaccatcatcaccaccatcacaacatcttcgagatgctccgtatcgacgagggcctcaggttgaagatctataaggacacagagggctattacaccatcggtattggtcatctcttgactaagtcgccttcacttaacgcagcaaaatccgaactggacaaggccatcggtcgcaatacaaatggtgtcattactaaggacgaagctgaaaagctcttcaaccaggacgttgatgcagctgttcgcggaatcctgcgcaacgcaaagctcaagccggtttacgattctttggacgccgttcgccgtgcggccttgattaacatggtgtttcagatgggtgaaaccggagttgcaggatttaccaactcgctgaggatgctgcaacaaaagagatgggacgaagccgctgtgaatctcgccaaatctaggtggtacaaccagacacccaatcgtgcgaagagagtgataactacattccgcaccggtacttgggacgcatatgaaaacctgtactttcaggggtccacactgcacggcctggcccacatcttctcctacgagcgcctgtctctgaagcgcgcactgtgggccctgtgcttcctgggctccctggctgtgctgctgtgtgtgtgcacggagcgcgtgcagtactacttccactaccaccatgtcaccaagctcgacgaggtggctgcctctcagcttaccttccctgctgtcacgctgtgcaacctcaacgagttccgctttagccaagtctccaagaatgacctgtatcatgctggcgagctgctggccctgctcaacaacaggtatgagataccagacacacagatggcagatgaaaagcagctggagatactgcaggacaaagccaacttccgcagcttcaaacccaaacccttcaacatgcgcgagttctacgaccgcgctggccacgacattcgcgacatgctgctctcctgccacttccgcggcgaggtctgcagcgctgaagacttcaaggtggtcttcacacgctatggaaagtgctacacgttcaactccggccgcgatggccgcccgcgcctgaagaccatgaagggtggcacgggcaatggcctggaaatcatgctggacatccagcaggacgagtacctgcctgtgtggggcgagactgacgagacgtccttcgaagcaggcatcaaagtgcagatccatagtcaggatgaacctcctttcatcgaccagctgggctttggcgtggccccaggcttccagacctttgtggcctgccaggagcagcgcctcatctacctgcccccaccctggggcacctgcaaagctgttaccatggactccgatttggatttcttcgactcctacagcatcactgcctgccgcatcgactgtgagacgcgctacctggtggagaactgcaactgccgcatggtgcacatgccaggcgatgccccatactgtactccagagcagtacaaggagtgtgcagatcctgctctggacttcctggtggaaaaggaccaggagtactgcgtgtgtgaaatgccttgcaacctgacccgctatggcaaagagctgtccatggtcaagatccccagcaaagcctcagccaagtacctggccaagaagttcaacaaatctgagcaatacataggcgagaacatcctggtgctggacattttctttgaagtcctcaactatgagaccattgaacagaagaaggcctatgagattgcaggcctcctgggtgacatcggcggccagatgggcctgttcatcggcgccagcatcctcacggtgctggagctctttgactacgcctacgaggtcattaagcacaagtaa

SEQ ID NO: 9 - protein, 580 aa, unknown
  Fusion expression of GLUT1 protein and BRIL protein
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKADLEDNWETLNDNLKVIEKADNAAQVKDALTKMRAAALDAQKATPPKLEDKSPDSPEMKDFRHGFDILVGQIDDALKLANEGKVKEAQAAAEQLKTTRNAYIQKYLEKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQVHHHHHHHHHH

SEQ ID NO: 10 - protein, 649 aa, unknown
  Fusion expression of GLUT1 protein and T4L protein
MNIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAYMEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTHHHHHHHHHH

SEQ ID NO: 11 - DNA, 1743 bp, unknown
  Fusion expression of GLUT1 protein and BRIL protein
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaaggctgatctggaagacaattgggaaactctgaacgacaatctcaaggtgatcgagaaggctgacaatgctgcacaagtcaaagacgctctgaccaagatgagggcagcagccctggacgctcagaaggccactccacctaagctcgaggacaagagcccagatagccctgaaatgaaagactttcggcatggattcgacattctggtgggacagattgatgatgcactcaagctggccaatgaagggaaagtcaaggaagcacaagcagccgctgagcagctgaagaccacccggaatgcatacattcagaagtacctggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgcaccaccatcaccaccatcaccaccatcattaa

SEQ ID NO: 12 - DNA, 1950 bp, unknown
  Fusion expression of GLUT1 protein and T4L protein
atgaacatcttcgagatgctccgtatcgacgagggcctcaggttgaagatctataaggacacagagggctattacaccatcggtattggtcatctcttgactaagtcgccttcacttaacgcagcaaaatccgaactggacaaggccatcggtcgcaatacaaatggtgtcattactaaggacgaagctgaaaagctcttcaaccaggacgttgatgcagctgttcgcggaatcctgcgcaacgcaaagctcaagccggtttacgattctttggacgccgttcgccgtgcggccttgattaacatggtgtttcagatgggtgaaaccggagttgcaggatttaccaactcgctgaggatgctgcaacaaaagagatgggacgaagccgctgtgaatctcgccaaatctaggtggtacaaccagacacccaatcgtgcgaagagagtgataactacattccgcaccggtacttgggacgcatatatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacaccaccatcaccaccatcaccaccatcattaa

SEQ ID NO: 13 - DNA, 69 bp, Unknown
  primer
cccaccatcgggcgcggatccatgcatcaccatcatcaccaccatcacgctgatctggaagacaattgg

SEQ ID NO: 14 - DNA, 42 bp, Unknown
  primer
cccctgaaagtacaggttttccaggtacttctgaatgtatgc

SEQ ID NO: 15 - DNA, 46 bp, Unknown
  primer
gaaaacctgtactttcaggggtccacactgcacggcctggcccaca

SEQ ID NO: 16 - DNA, 46 bp, Unknown
  primer
cttggtaccgcatgcctcgagttacttgtgcttaatgacctcgtag

SEQ ID NO: 17 - DNA, 66 bp, Unknown
  primer
cccaccatcgggcgcggatccatgcatcaccatcatcaccaccatcacgaaaacctgtactttcag

SEQ ID NO: 18 - DNA, 46 bp, Unknown; identical to SEQ ID NO: 16
  primer

SEQ ID NO: 19 - DNA, 69 bp, Unknown
  primer
cccaccatcgggcgcggatccatgcatcaccatcatcaccaccatcacaacatcttcgagatgctccgt

SEQ ID NO: 20 - DNA, 43 bp, Unknown
  primer
cccctgaaagtacaggttttcatatgcgtcccaagtaccggtg

SEQ ID NO: 21 - DNA, 46 bp, Unknown; identical to SEQ ID NO: 15
  primer

SEQ ID NO: 22 - DNA, 46 bp, Unknown; identical to SEQ ID NO: 16
  primer

SEQ ID NO: 23 - DNA, 66 bp, Unknown; identical to SEQ ID NO: 17
  primer

SEQ ID NO: 24 - DNA, 46 bp, Unknown; identical to SEQ ID NO: 16
  primer

SEQ ID NO: 25 - DNA, 51 bp, Unknown
  primer
cccgtttctgctagcaagcttaccatgaacatcttcgagatgctccgtatc

SEQ ID NO: 26 - DNA, 47 bp, Unknown
  primer
cagcttcttgctgctgggctccatatatgcgtcccaagtaccggtgc

SEQ ID NO: 27 - DNA, 24 bp, Unknown
  primer
atggagcccagcagcaagaagctg

SEQ ID NO: 28 - DNA, 53 bp, Unknown
  primer
atgatggtggtgatggtggtgatggtggtgtgtcttgtcactttggctggctc

SEQ ID NO: 29 - DNA, 51 bp, Unknown; identical to SEQ ID NO: 25
  primer

SEQ ID NO: 30 - DNA, 46 bp, Unknown
  primer
ggtcgaggtcgggggatccttaatgatggtggtgatggtggtgatg

SEQ ID NO: 31 - DNA, 26 bp, Unknown
  primer
gctgatctggaagacaattgggaaac

SEQ ID NO: 32 - DNA, 25 bp, Unknown
  primer
caggtacttctgaatgtatgcattc

SEQ ID NO: 33 - DNA, 48 bp, Unknown
  primer
cccgtttctgctagcaagcttaccatggagcccagcagcaagaagctg

SEQ ID NO: 34 - DNA, 48 bp, Unknown
  primer
gtttcccaattgtcttccagatcagccttggcccggttctcctcgttg

SEQ ID NO: 35 - DNA, 50 bp, Unknown
  primer
gaatgcatacattcagaagtacctggagaagaaggtcaccatcctggagc

SEQ ID NO: 36 - DNA, 50 bp, Unknown
  primer
atgatggtggtgatggtggtgatggtggtgcacttgggaatcagccccca

SEQ ID NO: 37 - DNA, 48 bp, Unknown; identical to SEQ ID NO: 33
  primer

SEQ ID NO: 38 - DNA, 46 bp, Unknown; identical to SEQ ID NO: 30
  primer